(19) United States
(12) Patent Application Publication
(10) Pub. No.: US 2019/0159942 A1
(43) Pub. Date: May 30, 2019

(54) DENTURE ADHESIVE REMOVAL PAD

(71) Applicant: Bettina E. Lucci, Lakewood, CO (US)

(72) Inventor: Bettina E. Lucci, Lakewood, CO (US)

(21) Appl. No.: 16/199,939

(22) Filed: Nov. 26, 2018

Related U.S. Application Data
(60) Provisional application No. 62/590,467, filed on Nov. 24, 2017.

(51) Int. Cl. A61F 13/45; A61F 13/15

(52) U.S. Cl. CPC A61F 13/45; A61C 17/00; A61F 2013/15008; A61F 13/15203

(57) ABSTRACT

A denture adhesive removal pad. The denture adhesive removal pad has a first portion and a second portion for absorbing denture adhesive. A narrowed connector portion is attached to both the first portion and the second portion and allows the denture adhesive removal pad to be folded. The shape of the first portion and the second portion can be identical or different.

### CROSS REFERENCE TO RELATED APPLICATIONS

This application claims the benefit of U.S. Provisional Application No. 62/590,467 filed on Nov. 24, 2017. The above identified patent application is herein incorporated by reference in its entirety to provide continuity of disclosure.

### BACKGROUND OF THE INVENTION

The present invention relates to a denture adhesive removal pad. Glues and adhesives that are designed for securing dentures to a user are commonly difficult to control. Once these substances become smeared and displaced around the mouth of the user, they can be difficult to remove, causing frustration or discomfort. In some cases, adhesives may induce gagging.

Many denture users try to use paper towels or toilet paper to wipe down their gums, mouth and dentures in the event of an accidental overuse of denture adhesive. However, these methods may be unsanitary as these materials, particularly when gathered from a public restroom, may be exposed to contaminants. In addition to this method being unsanitary, it is also ineffective. The structural stability of paper towels and toilet paper breaks down in the presence of moisture. This inherent characteristic of these paper products is exacerbated in the mouth of a user as saliva is excreted in the mouth.

Additionally, current methods may be time-consuming. Many users of dentures are hesitant to clean their dentures in an open environment for personal or hygienic reasons. When an individual must remove excess adhesive from their mouth, it may appear unsightly to observers. As such, a user may desire to clean their dentures in a bathroom or other area that provides privacy, however such facilities are not always readily available.

As such, there is a need defined in the known arts for a denture adhesive removal pad that enables a user to quickly, effectively and discreetly remove an excessive amount of denture adhesive disposed in a mouth of a user.

### SUMMARY OF THE INVENTION

In view of the foregoing disadvantages inherent in the known types of removal pads now present in the known art, the present invention provides a denture adhesive removal pad wherein the same can be utilized for providing convenience for the user when removing an excess portion of denture adhesive from his or her mouth.

The present system comprises a fabric member, capable of absorbing a substance, such as a dental adhesive. The fabric member is flexible. Furthermore, the fabric member has a first side and a second side. The first side and the second side each comprise an absorbent material, such that the dental adhesive can be absorbed therein. Additionally, the fabric member defines a first portion and a second portion such that a user can use the denture adhesive removal pad more than once. A connector is disposed between the first portion and the second portion.

In another embodiment, at least one of the first portion and the second portion define at least one corner thereon, such as to improve functionality for the user.

In a further embodiment, each of the first portion and the second portion are identical in size and shape such that one of the first side or the second side is protected when the dental adhesive removal pad is placed into a folded configuration.

In yet another embodiment, each of the first portion and the second portion define a rounded edge opposite a squared edge.

In yet a further embodiment, the first portion is circular in shape and the second portion defines a rounded edge opposite a squared edge.

In another embodiment, the first portion is triangular in shape and the second portion defines a rounded edge opposite a squared edge.

In a further embodiment, the first portion is a square shape and the second portion defines a rounded edge opposite a squared edge.

In yet another embodiment, the first portion is a square shape and the second portion defines a square shape.

In yet a further embodiment, the connector portion is made of a flexible material such as to provide convenience and ease of use to a user.

In another embodiment, a first fabric member and a second fabric member are symmetrical and sewn together around a shared perimeter such that the absorbency of the denture adhesive removal pad is increased.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the denture adhesive removal pad.

FIG. 2 shows a front view of an embodiment of the denture adhesive removal pad.

FIG. 3 shows a front view of an alternate embodiment of the denture adhesive removal pad.

FIG. 4 shows a front view of another embodiment of the denture adhesive removal pad.

FIG. 5 shows a front view of yet another embodiment of the denture adhesive removal pad.

FIG. 6 shows a perspective view of an embodiment of the denture adhesive removal pad in use.

### DETAILED DESCRIPTION OF THE INVENTION

Reference is made herein to the attached drawings. Like reference numerals are used throughout the drawings to depict like or similar elements of the denture adhesive removal pad. The figures are intended for representative purposes only and should not be considered to be limiting in any respect.

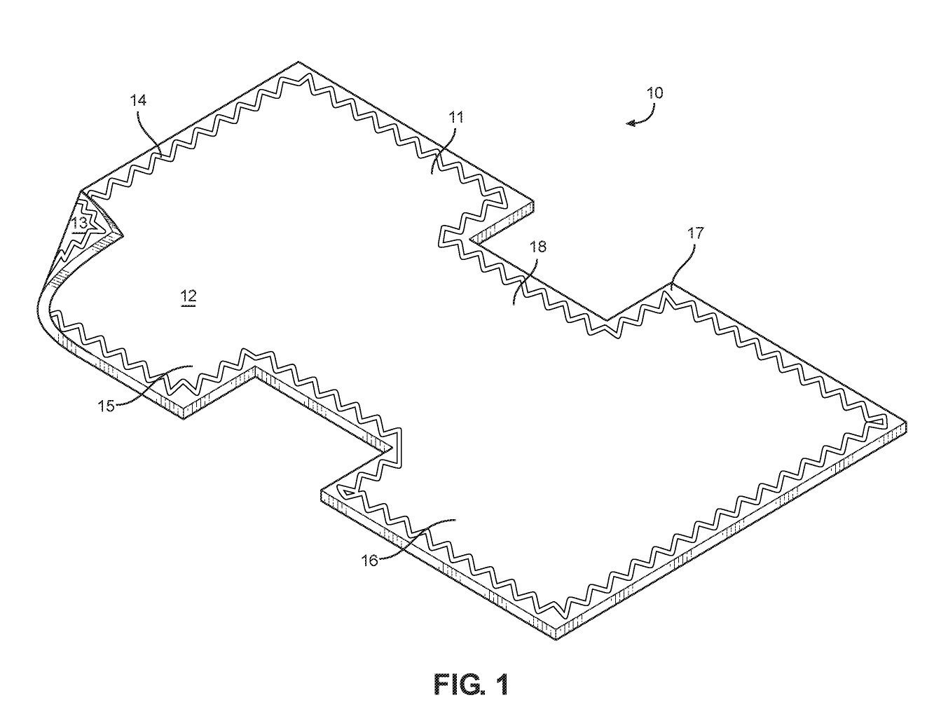

Referring now to FIG. 1, there is shown a perspective view of an embodiment of the denture adhesive removal pad. The denture adhesive removal pad 10 comprises a fabric member 11. The fabric member 11 is made of a flexible material. The flexible material enables the user to contour the denture adhesive removal pad 10 such as to effectively gather an excess portion of denture adhesive therein.

The fabric member 11 has a first side 12 and a second side 13. In one embodiment, the fabric member 11 comprises a first fabric member attached to a second fabric member around a perimeter thereof. In the shown embodiment, the first fabric member is attached to the second fabric member via a string 14. In this way, the first side 12 and the second side 13 comprises the first fabric member and the second fabric member, respectively.

The first side 12 of the fabric member 11 comprises an absorbent material thereon. In another embodiment, the second side 13 of the fabric member comprises an absorbent material thereon. The absorbent material is any material suitable for absorbing an excess portion of denture adhesive. In one embodiment, the absorbent material is a multi-layer absorbent material, such as gauze.

In one embodiment, the fabric member 11 is comprises of a first fabric member and a second fabric member. The first fabric member is attached around a perimeter thereof to the second fabric member, such that a housing is formed therebetween. The housing allows for additional absorption of denture adhesive when the denture adhesive removal pad 10 is in use.

Furthermore, the fabric member 11 defines a first portion 15 and a second portion 16. The first portion 15 is disposed opposite of the second portion 16. In the shown embodiment, the first portion 15 and the second portion 16 are identical in size and shape. When the first portion 15 and second portion 16 are identical in size and shape, the user may place the denture adhesive removal pad 10 into a folded configuration such that either the first side 12 or the second side 13 is protected from contaminants. When in the folded configuration, a perimeter of the first portion 15 rests flush with a perimeter of the second portion 16, thereby minimizing form factor of the denture adhesive removal pad 10 for storage, while also protecting an operative side of the first and second portions 15, 16 from exposure to external contaminants.

In the shown embodiment, each of the first portion 15 and the second portion 16 define a square shape. Each square shape defines four corners 17. The corners 17 provide convenience to a user in maneuvering the denture adhesive removal pad 10 in the mouth of the user. For example, a corner 17 may be used to absorb an excess portion of denture adhesive from a space between two teeth.

The first portion 15 is connected to the second portion 16 via a connector portion 18. The connector portion 18 is of a lesser width than each of the first portion 15 and the second portion 16, such as to provide clear definition of the first portion 15 and the second portion 16. Additionally, the connector portion 18 is flexible, such as to enable efficient maneuverability of each of the first portion 15 and the second portion 16. Furthermore, flexibility of the connector portion 18 enables placement of the denture adhesive removal pad 10 into a folded configuration.

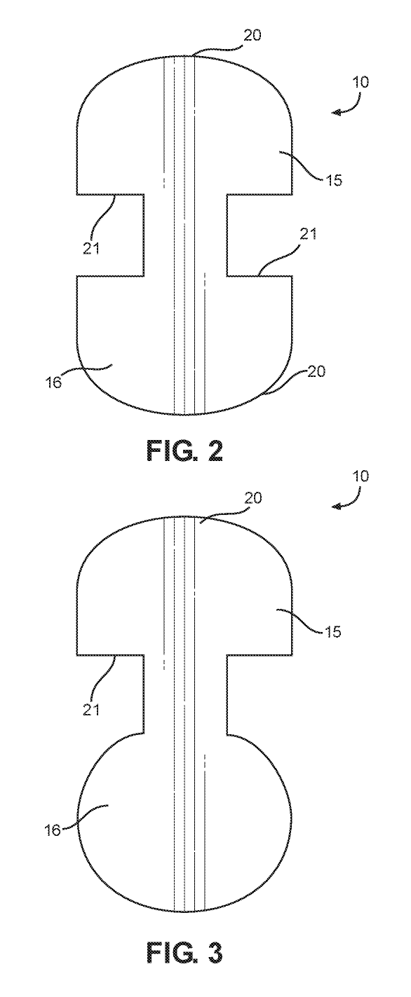

Referring now to FIG. 2, there is shown a front view of an embodiment of the denture adhesive removal pad. In the shown embodiment of the denture adhesive removal pad 10, each of the first portion 15 and the second portion 16 defines a portion having a rounded edge 20 opposite of squared edge 21. The squared edge 21 provides a pair of corners. The corners provide convenience to a user in maneuvering the denture adhesive removal pad in the mouth of the user. Additionally, the rounded edge 20 provides an absence of corners, such as to provide functionality to a user where utilizing the denture adhesive removal pad in a location where a corner may cause irritation, such as the tongue or throat of the user. In one embodiment, the width of the first and second portions are the same on opposing sides of the connector portion.

Referring now to FIG. 3, there is shown a front view of an embodiment of the denture adhesive removal pad. In one embodiment, the first portion 15 and the second portion 16 are of a uniform configuration. In another embodiment, the first portion 15 and the second portion 16 are of a non-uniform configuration. In the shown embodiment, of the denture adhesive removal pad 10, the first portion 15 defines a rounded edge 20 opposite a squared edge 21.

Furthermore, the second portion 16 is circular in shape. The circular shape of the second portion 16 allows a user the advantages of having a portion 16 of the dental adhesive removal pad 10 that is entirely devoid of corners. Such a configuration may be desired by a user where denture adhesive must be removed from a sensitive area of the mouth of the user, such as the throat or underneath the tongue.

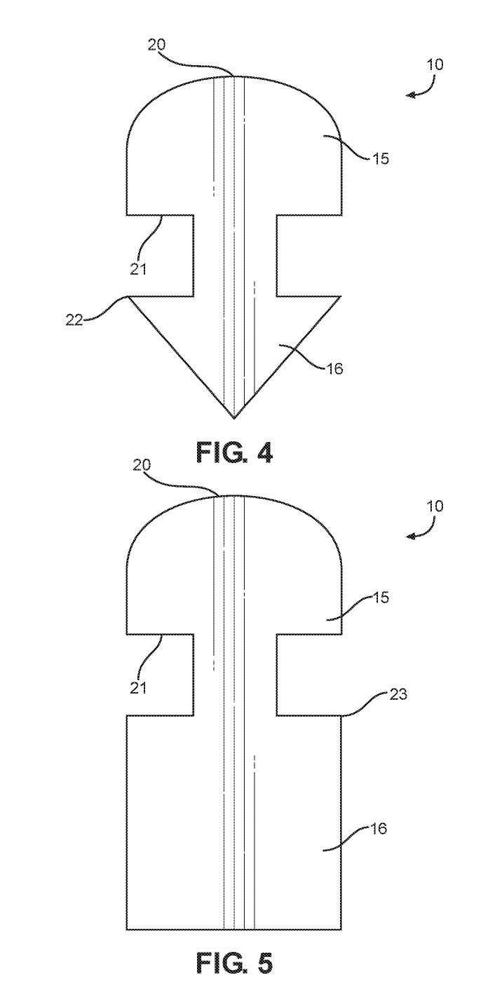

Referring now to FIG. 4, there is shown a front view of an embodiment of the denture adhesive removal pad. In the shown embodiment of the denture adhesive removal pad 10, the first portion 15 defines a rounded edge 20 opposite a squared edge 21. The second portion 16 is triangular in shape. When the second portion is triangular in shape, three angular sections 22 are provided. The angular sections 22 are of any desired angle. The angular sections 22 configured to allow the user to remove dental adhesive from a narrow area, such as between teeth.

Referring now to FIG. 5, there is shown a front view of an embodiment of the denture adhesive removal pad. In the shown embodiment, of the denture adhesive removal pad 10, the first portion 15 defines a rounded edge 20 opposite a squared edge 21. Furthermore, the second portion 16 defines four corners 23 thereon wherein each corner is of a uniform angle.

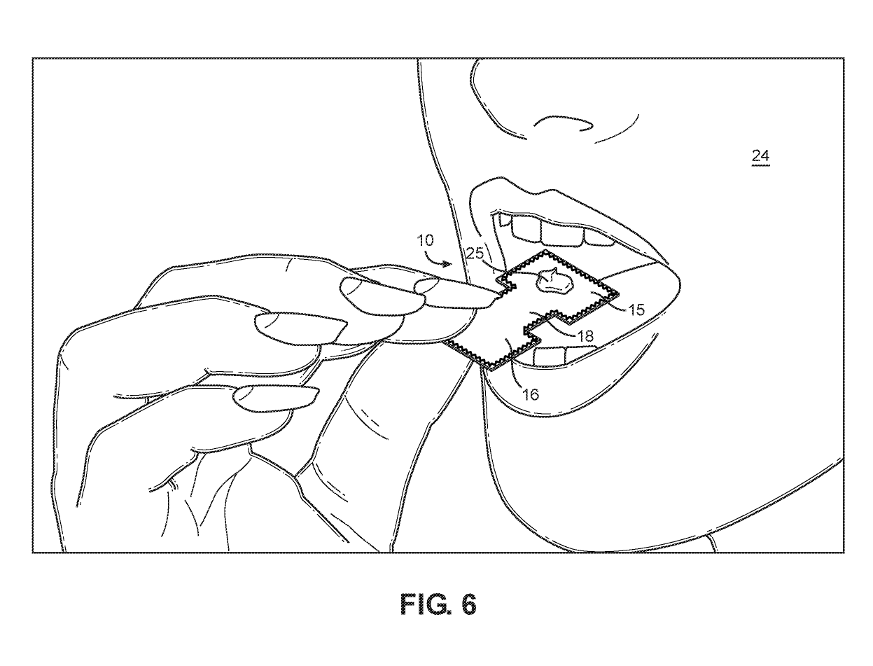

Referring now to FIG. 6, there is shown a perspective view of an embodiment of the denture adhesive removal pad in use. In use, the user will remove the denture adhesive removal pad 10 from a stored location. The user will orient the denture adhesive removal pad 10 into an unfolded configuration. The unfolded configuration is defined where the first portion 15 is disposed oppositely the second portion 16 wherein the narrowed connector portion 18 is extended. Either the first portion 15 or the second portion 16 is inserted into the mouth of the user 24. The first portion 15 and the second portion 16 will absorb an excess portion of denture adhesive 25 disposed in the mouth of the user. Upon removal of the denture adhesive removal pad 10 from the mouth of the user 24, the denture adhesive removal pad 10 can be returned to a folded configuration wherein the first portion 15 and the second portion 16 are placed into contact with each other and the narrowed connector portion 18 is folded over itself.

Alternatively, in the event of an amount of denture adhesive disposed in the mouth exceeding an amount absorbable by the first portion 15 or the second portion 16 alone, both the first portion 15 and the second portion 16 can be used to absorb the denture adhesive. The first portion 15 can absorb a first amount of denture adhesive and the second portion 16 can absorb a second amount of denture adhesive.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A denture adhesive removal pad, comprising:
a fabric member;
the fabric member made of a flexible material;
the fabric member having a first side and a second side;
wherein the first side comprises an absorbent material thereon;
wherein the second side comprises an absorbent material thereon;
the fabric member defining a first portion and a second portion, wherein a narrowed connector portion extends therebetween.

2) The denture adhesive removal pad of claim 1, wherein each of the first portion and the second portion define a plurality of corners thereon.

3) The denture adhesive removal pad of claim 1, wherein each of the first portion and the second portion are identical in size and in shape.

4) The denture adhesive removal pad of claim 1, wherein each of the first portion and the second portion define a rounded edge opposite a squared edge.

5) The denture adhesive removal pad of claim 1, wherein the first portion is circular in shape and the second portion defines a rounded edge opposite a squared edge.

6) The denture adhesive removal pad of claim 1, wherein the first portion is triangular in shape and the second portion defines a rounded edge opposite a squared edge.

7) The denture adhesive removal pad of claim 1, wherein the first portion is a square shape and the second portion defines a rounded edge opposite a squared edge.

8) The denture adhesive removal pad of claim 1, wherein the first portion is a square shape and the second portion is a square shape.

9) The denture adhesive removal pad of claim 1, wherein the connector portion is flexible.

10) A denture adhesive removal pad, comprising:
a housing, wherein the housing is formed by a first fabric member attached around a perimeter thereof to a second fabric member at a corresponding perimeter thereof;
the first fabric member and the second fabric member made of a flexible material;
the first fabric member and the second fabric member made of an absorbent material;
the fabric member defining a first portion and a second portion, wherein a narrowed connector portion extends therebetween.

11) The denture adhesive removal pad of claim 10, wherein each of the first portion and the second portion define a plurality of corners thereon.

12) The denture adhesive removal pad of claim 10, wherein each of the first portion and the second portion are identical in size and in shape.

13) The denture adhesive removal pad of claim 10, wherein each of the first portion and the second portion define a rounded edge opposite a squared edge.

14) The denture adhesive removal pad of claim 10, wherein the first portion is circular in shape and the second portion defines a rounded edge opposite a squared edge.

15) The denture adhesive removal pad of claim 10, wherein the first portion is triangular in shape and the second portion defines a rounded edge opposite a squared edge.

16) The denture adhesive removal pad of claim 10, wherein the first portion is a square shape and the second portion defines a rounded edge opposite a squared edge.

17) The denture adhesive removal pad of claim 10, wherein the first portion is a square shape and the second portion is a square shape.

18) The denture adhesive removal pad of claim 10, wherein the connector portion is flexible.

19) A method of removing a denture adhesive from a mouth of a user, comprising:
removing a denture adhesive removal pad from a stored location;
arranging the denture adhesive removal pad into an unfolded configuration;
inserting the denture adhesive removal pad into the mouth of the user;
absorbing an excess portion of denture adhesive disposed in the mouth into the denture adhesive removal pad;
removing the denture adhesive removal pad from the mouth;
disposing of the denture adhesive removal pad.

20) The method of removing a denture adhesive from a mouth of a user of claim 19, further comprising:
absorbing a first portion of a denture adhesive disposed in the mouth into a first portion of the denture adhesive removal pad;
absorbing a second portion of the denture adhesive disposed in the mouth using a second portion of the denture adhesive removal pad.
